(19) United States
(12) Patent Application Publication
(10) Pub. No.: US 2018/0092768 A1
(43) Pub. Date: Apr. 5, 2018

(54) Foreskin Condom

(71) Applicant: Mazy Holiday, Birmingham, AL (US)

(72) Inventor: Mazy Holiday, Birmingham, AL (US)

(21) Appl. No.: 15/285,135

(22) Filed: Oct. 4, 2016

(51) Int. Cl. A61F 6/04

(52) U.S. Cl. CPC A61F 6/04; A61F 2006/048

(57) ABSTRACT

A prophylactic condom for simulating the sensation and natural lubricating properties of an uncircumcised penis includes an open shaft section which is elastic as to hold and encase a penis of varying size and shape of and a distal head section having a wide diameter bulbous portion intended to fold down around the shaft section during sexual intercourse. The bulbous portion forms an external sleeve in which the shaft section and encased penis may move into and out of during a sexual encounter without the necessity of additional lubricant or risking the occurrence of a breach of the condom due to the frictions involved in intercourse.

### FIELD OF THE INVENTION

The herein disclosed invention relates to a condom for the purpose of preventing unwanted pregnancy and prevention of the transmission of sexually transmitted diseases during sexual intercourse, and more specifically to a unique and novel condom head and shaft implement for simulating the feeling that the male is uncircumcised which has features more comfortable and pleasurable for both men and woman.

### BACKGROUND

Generally a condom is comprised of a thin membrane in the form of a rolled tubular sheath, which is unrolled and worn over the penis as a prophylactic barrier to prevent or guard against pregnancy and/or transmission of sexually transmitted diseases during intercourse. The barrier membrane of a typical condom is made of a liquid emulsion of latex or similar elastomeric materials formed to provide a thin membrane for optimizing sensitivity, tactile sensation, and heat transference from a penis during intercourse while still maintaining the strength and prophylactic properties of the condom. Recognizing that the thinnest barrier membrane will enhance the sensitivity, tactile sensation, and heat transference of the condom, the risk of a break or tear in a condom membrane during sexual intercourse is always a significant concern in condom design and manufacturing.

Generally, condoms are manufactured utilizing a dipping process, whereby an appropriately dimensioned mold or mandrel is dipped into a latex rubber emulsion, for example, formulated with various curing (vulcanizing) agents and accelerators, to create the condom membrane. The mold covered with the latex rubber is then withdrawn from the emulsion and subjected to elevated temperatures for drying and curing of the latex rubber membrane. In doing so the condom achieves the desired physical properties. The cured condom is then stripped from the mold and rolled into a toroidal configuration for packaging. Prior to intercourse, the condom is donned by placing the condom onto the head of a penis and unrolling it and extending the condom membrane downward along the shaft of the penis.

Some condoms are manufactured with cross ribs or other projections along the internal or external surfaces of a condom membrane for the purpose of stimulating the male and female during sexual intercourse. It was believed that the textured surface of such condoms provides increased frictional and tactual kinetics for enhancing tactile sensation and stimulation. Typically, the ribs or projections are integrally formed on the membrane surface of the condom during manufacture by the use of grooves or notches etched in the surface of the mold during the dipping process. The design of such ribs and projections range from interrupted rings canted with respect to the radial plane, helical and honeycomb patterns, and other various ribbed arrangements.

The problem with condoms having such ribs and projections is that the added texture of the ribs and projections may be abrasive or an irritant to a partner's tender areas. Contrary to the intent of such ribbed condoms, many partners prefer the natural feel of the male penis, and, in particular, that of the uncircumcised penis which serves as a natural lubricant. Intercourse with a man having a circumcised penis or where the man wears a traditional condom, all the lubrication needed for sexual intercourse is left to be provided naturally by a partner or a commercial lubricant. The inability to provide natural lubrication may lead to problems initiating, progressing, and concluding intercourse. Failing to use a commercial lubricant to maintain lubrication during intercourse may also impose the risk of pain or breach of the condom due to the pressures and frictional forces occurring during intercourse.

Yet another problem is that the configuration of the grooves or notches forming the ribs or projections on the mold during the dipping step of the manufacturing process previously described contributes to breakages or tears in condoms during use, defeating the prophylactic purpose of preventing pregnancy and transmission of sexually transmitted diseases. When a smooth mold is withdrawn from a latex rubber emulsion, the uncured liquid latex rubber flows downward, ideally forming a uniform coating or layer of the rubber emulsion around the mold. However, as the uncured latex rubber flows along the surface of a grooved or notched mold, the grooves or notches in the mandrels interfere with the uniform flow of the rubber emulsion causing potential backfill problems which can result in the formation of undesirable weak spots in the condom and non-uniformity in wall thickness of the condom membrane.

It would therefore be a significant advance in the art of condoms to develop a condom which simulates and enhances the feeling of the natural lubricating properties of an uncircumcised penis to a sexual partner while avoiding the abrasiveness of ribbed condoms and preventing the weak spots and the resulting membrane failures that can be associated with prior art ribbed/textured condoms. An additional benefit of the condoms of the present invention is that these condoms are capable of being fabricated in a cost efficient and effective manner, using known conventional methods of condom production. This enables manufacturing facilities to produce the improved condoms with minimal retooling and/or changes to existing equipment.

### SUMMARY OF THE INVENTION

The present invention is generally directed to a condom comprised of a continuous elastic tubular membrane having a shaft section, an open proximal end and a closed distal end. The diameter of the tubular elastic tubular membrane widens along an area between the distal and the proximal end of the condom to create an integral cover for the shaft section of the tubular membrane. The cover is intended to fold or collapse down over the shaft section toward the open proximal end of the condom forming an external sleeve in which the penis may move within during sexual intercourse. In particular, the external sleeve created by the cover due to the widened diameter of the tubular membrane mimics a foreskin and recreates the natural lubricating characteristics found in an uncircumcised penis, while maintaining the prophylactic qualities of the condom. In use, the sleeve created by the cover will allow for stimulation of a sexual partner through the natural contour and tactual kinetics of the penis without the abrasive characteristics of prior art ribbed condoms.

The condom of the present invention is formed by providing a flow of an emulsion of latex or similar elastomeric material over a mold having rounded smooth surfaces. The rounded smooth surfaces of the mold will prevent a backup in the flow of the emulsion of elastomeric material and promote the even distribution of the emulsion over the mold, wherein, after being dried, the emulsion will create a barrier membrane for the condom will be of sound integrity for use in sexual intercourse.

These and other objects, advantages, and features of this invention will be apparent to those skilled in the art from a consideration of this specification, including the claims and drawings herein.

### BRIEF DESCRIPTION OF THE DRAWINGS

FIG. 1 is a side view of the foreskin condom of the present invention.

FIG. 2a is an end view of the closed distal head of the condom shown in FIG. 1.

FIG. 2b is an end view of the open proximal end of the condom shown in FIG. 1.

FIG. 3 is a side view of the condom of FIG. 1 with size dimensions indicated.

FIG. 4 is a side view of the condom of FIG. 1 in a flattened condition with the associated size dimensions indicated.

FIG. 5 is a cross section view of the condom of FIG. 1 in an inserted position.

FIG. 6a is a side view of the mold for use in manufacturing the condom of FIG. 1 in its expanded condition.

FIG. 6b is a side view of the mold for use in manufacturing the condom of FIG. 1 in its collapsed condition.

### DESCRIPTION OF THE INVENTION

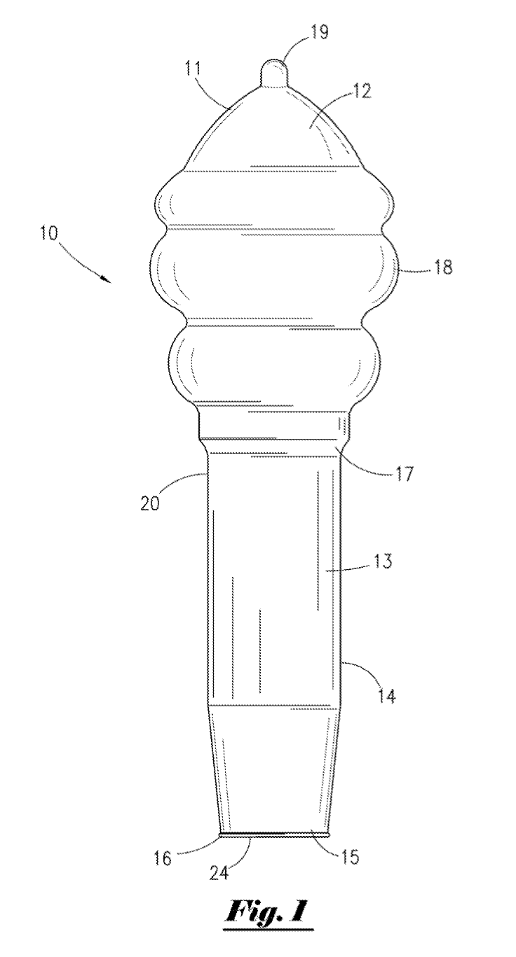

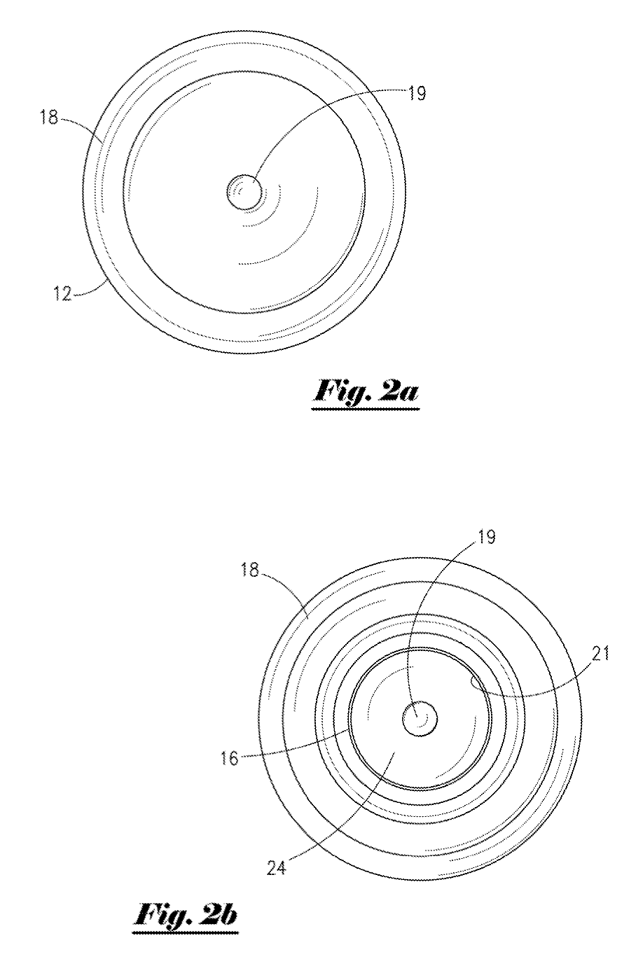

Referring now to the drawings and, more particularly to FIGS. 1, 2a, and 2b, there is shown the prophylactic foreskin condom 10 configured for simulating the properties and advantages of an uncircumcised penis. Prophylactic foreskin condom 10 is formed as an elastic tubular membrane 20, having a closed distal end 11, a head 12 adjacent closed distal end 11, an open proximal end 15 with central opening 24, and a shaft section 13 forming a tubular sheath 14. A nipple-shaped reservoir 19 may also be included at head 12 ejaculated semen may be collected and retained.

The membrane 20 of condom 10 has an outer surface 22 preferably formed of a homogenous layer of resilient prophylactically suitable liquid-impermeable material. Preferably the homogenous layer of resilient prophylactically suitable liquid-impermeable material will be formed of latex, polyurethane, or polyisoprene and be elastic to form around the varying size and shape of the penis P while remaining robust enough to withstand tearing under the pressures and frictions involved in sexual intercourse. The membrane 20 of condom 10 will preferably have a thickness in the range of about 0.002 to 0.006 inches. It can, however, be much thicker and consequently stronger than an ordinary condom without adversely affecting the enjoyment of either the male or female partner.

Shaft 13 is relatively tube shaped around the central opening 24 to provide entry of a penis into the elongate sheath 14. A beaded edge 16 is provided at end 15 around the central opening 24 upon which condom 10 may be unrolled. The sheath 14 of condom 10 is formed to provide tight fit on penis P as to make a seal for preventing leakage during normal use. The sheath 14 has a neck section 17 from which the membrane 20 extends to create a bulbous portion 18 on the outer surface 22 of the membrane 20 along the head 12 or along the sheath 14 of the condom 10, the bulbous portion 18 having a diameter greater than the diameter of the head 12 and the sheath 14.

The head 12 of the condom 10 is substantially conical in shape with the reservoir 19 located centrally at the closed distal end 11. The interior surface 21 of the tubular sheath 14 is smooth and terminates distally at the reservoir 19. The beaded edge 16 is rolled at the open proximal end 15 of the condom 10 for preventing tears.

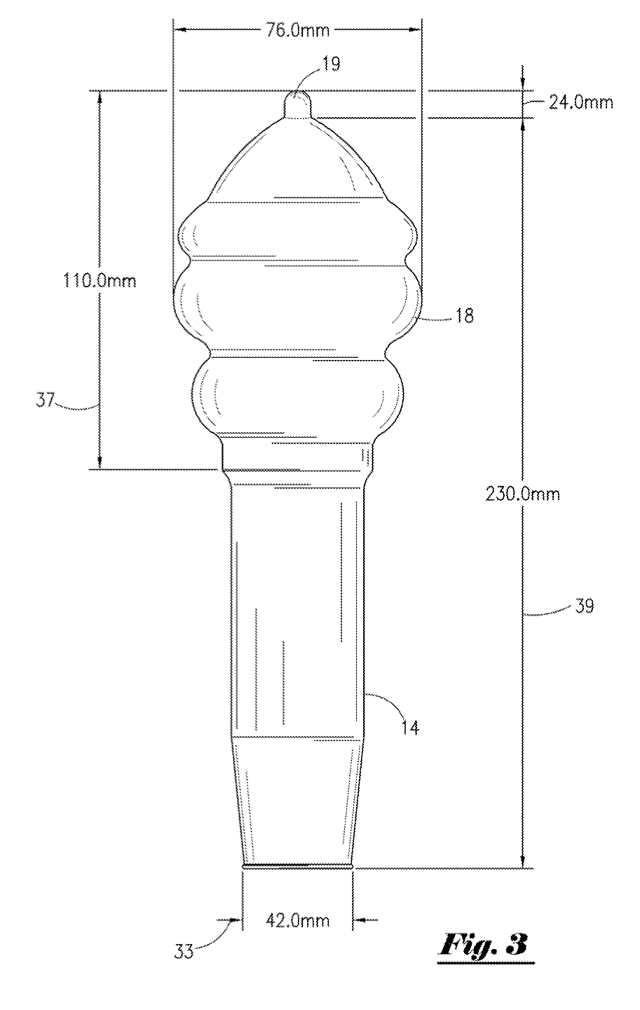

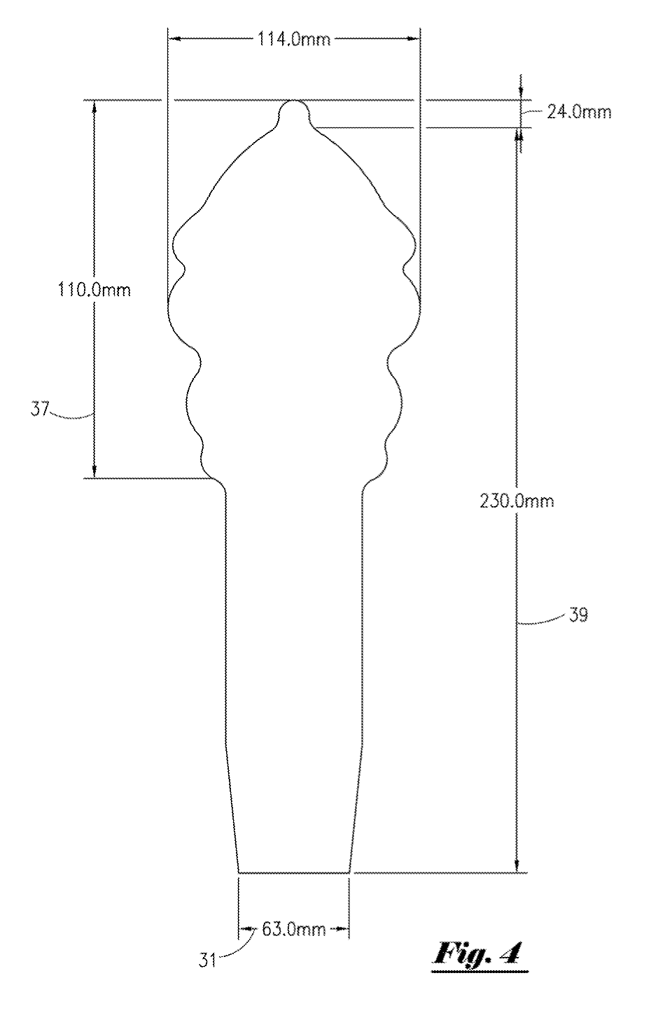

The exemplary dimensions of the condom 10 when expanded are shown in FIGS. 3 and 4. FIG. 3 shows the inner diameter and length of the condom 10 as indicated by arrows 33 and 39, respectively, and the preferable length of the reservoir 19 and the bulbous portion 18 as indicated by arrow 37. However, it will be understood that the bulbous portion 18 may begin and end at any point the shaft section 13 of the condom 10. FIG. 4 shows the same condom 10 in a flat, contracted condition with a width of 63 mm as indicated by arrow 31. These dimensions are shown for illustrative purposes and are not to be construed as limiting in scope. Other sizes, dimensions and pattern area coverage may be utilized as required for making condoms of different sizes and/or shapes.

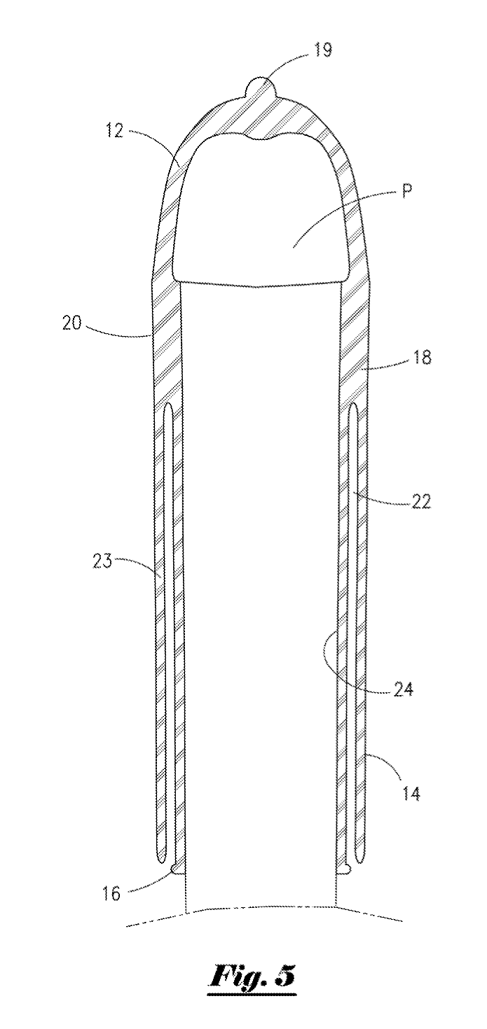

Now referring to FIG. 5, a cross section view of condom 10 is shown in an inserted position upon penis P. It is intended that during insertion of a penis P with condom 10 during intercourse, the expanded bulbous portion 18 on the outer surface of the condom 10 at the head 12 will fold downward at neck section 17 toward the proximal end 15 of the condom to cover sheath 14. This folded bulbous portion 18 of the membrane 20 creates a sleeve 23 around the sheath 14 of membrane 20 in which the sheath 14 with the enclosed penis P may move into and out of during intercourse and simulates the foreskin and natural lubricating properties of an uncircumcised penis. Use of the condom 10 will reduce or minimize the need for additional lubricants during the sexual encounter.

Use of the condom 10 will enhance pleasurable sensation and sensual excitement for both male and female partners. The sheath 14 and sleeve 23 configuration of condom 10 mimics an uncircumcised penis, increasing the tactual kinetics between the partners, and maintains the elastic grip of the sheath 14 to retain penis P within condom 10. It is envisioned that the bulbous portion 18 of the condom 10 will be sufficient to form sleeve 23 over almost the full length of sheath 14 to allow for full insertion of the penis P within sleeve 23. However, the dimensions of the bulbous portion 18 and the length of sleeve 23 are not limited as such and may be varied as required.

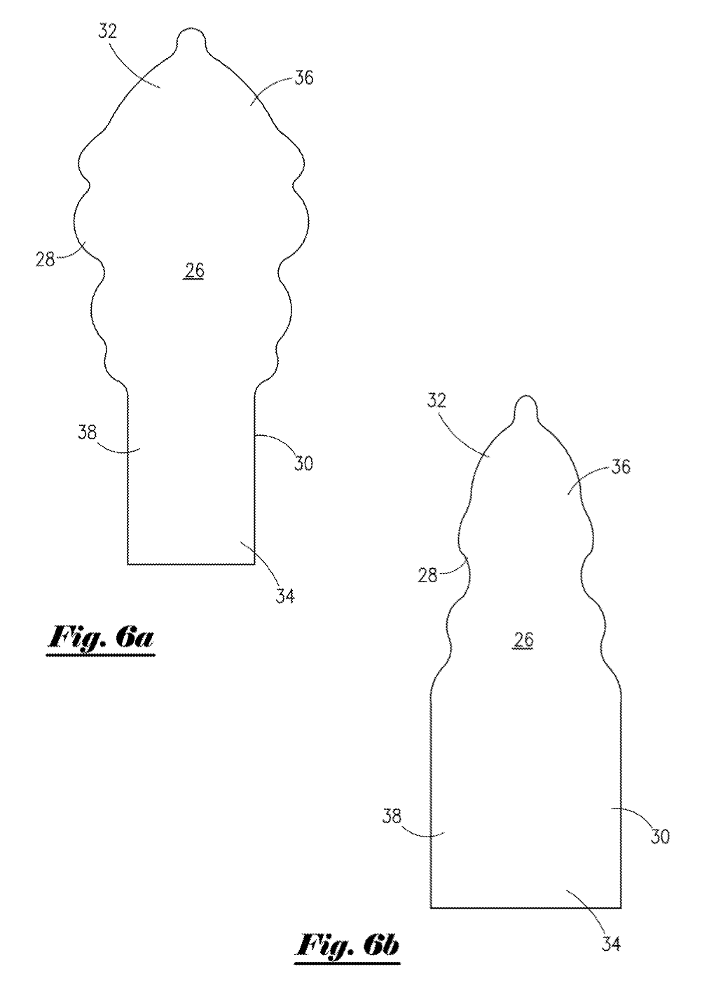

Referring to FIGS. 6a & 6b, a collapsible mold 26 for forming condom 10 of the present invention is shown. The mold 26 has and outer surface 30, a distal end 32, a proximal end 34, and a head section 36 and a shaft section 38 that correspond, respectively, with the head 12 and the shaft section 13 of a condom 10 of a desired configuration. A plurality of radial protuberances 28 are formed on the outer surface 30 for forming the bulbous portion 18 of condom 10. The shape of protuberances 28 may be adjusted as desired to create a desired corresponding bulbous portion 18. Preferably, the width of protuberances 28 on the outer surface 30 of mold 26 will be formed to provide a bulbous portion 18 sufficient to create a sleeve 23 on the condom 10 to cover sheath 14 during sexual activities. The protuberances 28 may be shaped in the form of succeeding waves to give the final impression of gentle folds in the membrane 20 or the protuberances 28 may be formed as a single bulb to create a smooth uniform bulbous portion 18. Additionally, it must be understood that protuberances 28 may begin and end anywhere along the shaft section 13 of the mold 26 depending on the desired configuration of the condom 10 to be formed. Additionally, it is, of course, possible to provide bulbous portions 18 of increased widths and heights by increasing the size of the protuberances 28 formed into mold 26.

The membrane 20 of the condom 10 having sheath 14 and bulbous portion 18 are simultaneously provided in the dipping process. Mold 26 is preferably collapsible to allow for the removal of condom 10 from mold 26 without the risk of damage to membrane 20 by rolling over the wider diameter of distal end 32, which could cause stretching and possible compromise of the membrane 20. As can be seen in FIG. 6a, the mold 26 is shown in its expanded condition. Mold 26 is comprised of silicone, rubber, plastic, or other material conventional in the art capable of providing a collapsible mold surface. Condom 10 is formed by the dipping of the expanded mold 26 into a liquid emulsion of elastomeric material such as latex or other prophylactically suitable elastomeric material. When the mold is so dipped the emulsion of elastomeric material will tend to adhere to the mold outer surface 30, and flow along the surface to provide a uniform coating which in the end after drying forms the membrane 20 of the condom 10. In order to provide the bulbous portion 18 of a desired diameter, a protuberance 28 corresponding to the desired bulbous portion 18 is provided on the mold. Accordingly, the size of the mold recesses corresponds to the bulbous portion 18 which will be present in the completed condom 10.

A condom 10 is formed by successive immersions of the mold 26 with the distal end 32 first into the emulsion of elastomeric material with desired prophylactic properties. It is important that during the first immersion of mold 26 into the emulsion of elastomeric material that the resulting coating of elastomeric material be even and wafer thin and evenly distributed on the mold 26 so that the resulting inside or interior surface of the condom 10 is smooth and uniform to enhance the prophylactic properties of the resulting membrane 20. After running the mold 26 and the first coating of elastomeric material through a drying process, such as by passing the mold 26 through a drying oven, the mold 26 and the dried membrane of elastomeric prophylactically suitable material is generally dipped again to generate a condom 10 with a membrane 20 of the desired thickness. To produce a condom 10 with a membrane 20 of an adequate thickness and with prophylactically suitable properties, it is preferable that every condom 10 be first immersed and then subsequently cured with heat at least twice. The beaded edge 16 is formed by rolling the open end 15 down along the outside of surface 20.

The condom 10 is then leached, washed, and coated with powder. Once leached, washed, and coated, the mold 26 is compressed into its collapsed form, seen in FIG. 6b, wherein the finished condom 10 may be safely removed from mold 26 and turned inside out so that the bulbous portion 18, which was initially molded on the interior surface, is on the outside before being rolled for packaging. The condom 10 may be packaged dry, and lubricated with a coating of lubricant such as silicone or an appropriate anti-friction agent such as a water-based lubricant, and/or coated with a spermicidal agent for added protection against pregnancy. Note that the condom 10 can also be made from an emulsion of any suitable prophylactically resilient elastomeric material, such as natural rubber, synthetic rubber, latex, polyurethane, polyisoprene, polyurethane, neoprene, or combinations thereof, through use of the appropriate dipping solutions and technology. As well, though it is preferred that mold 26 be collapsible, mold 26 may also be formed of a non-collapsible stainless steel, aluminum, plastic, or glass, as conventional in condom manufacture, wherein the membrane 20 may be rolled off after production.

Typically, with prior art ribbed/textured condoms, the etched recesses in the mold for forming the ribbing or projections on the condom, interferes with the normal flow of uncured latex rubber over the mandrel surface generating areas of backfills and uneven distribution. This backfilling and uneven latex distribution during the dipping process eventually results in the formation weak areas in the surface of the cured condom. The resulting weak areas are susceptible to rupture and/or tearing. However, the protuberances 28 of the present invention, formed on mold 26 in smooth curves upon outer surface 30, without the edges commonly etched into molds to form ribs on prior art condoms, allows for a smooth flow of the emulsion of elastomeric prophylactically suitable material over the outer surface 30 of the mold 26 minimizing the flow disruption of the uncured emulsion of elastomeric material to provide an even distribution of the emulsion of elastomeric material over the mold 26 minimizing areas of backfill and thereby reducing or eliminating weak areas in the resulting condom 10.

Although the preferred embodiment of the invention has been shown and described, it is not intended to be limiting. Those of skill in the art may recognize various modifications to the embodiment, which modifications are meant to be covered by the spirit and scope of the appended claims and are not intended to be construed to be limiting to such and may include variations in sizes, shapes, areas having the bulbous portion 18, and other configurations.

I claim:

1. A prophylactic condom, comprising:
a. an elastic tubular membrane having a closed distal end and an open proximal end;
b. a bulbous portion on said tubular membrane that extends along at least a portion of said tubular membrane between said distal end and said proximal end, said bulbous portion having a diameter greater than the diameter of said tubular membrane.

2. The prophylactic condom recited in claim 1, wherein:
a. a sheath is formed by said tubular membrane from said open proximal end of said tubular membrane; and
b. whereby said bulbous portion forms a sleeve around said sheath upon insertion of a penis into said sleeve.

3. The prophylactic condom recited in claim 2, further comprising a reservoir at said closed distal end whereby ejaculated semen is collected and contained.

4. The prophylactic condom recited in claim 3, further comprising a neck section on said sheath between said open proximal end and said bulbous portion, whereat said bulbous portion forms said sleeve over said sheath.

5. The prophylactic condom recited in claim 4, further comprising a beaded edge around said tubular membrane at said open proximal end whereby said tubular membrane may be rolled and unrolled.

6. The prophylactic condom recited in claim 5, wherein said tubular membrane is comprised of resilient elastomeric material.

7. The prophylactic condom recited in claim 6, wherein said resilient elastomeric material includes rubber, latex, polyurethane, polyisoprene, polyurethane, or neoprene.

8. The prophylactic condom recited in claim 7, further comprising a coating of lubricant over said tubular membrane.

9. The prophylactic condom recited in claim 3, wherein said sheath formed by said tubular membrane and said sleeve formed by said bulbous portion are formed integrally with said tubular membrane.

10. The prophylactic condom recited in claim 9, wherein said tubular membrane is comprised of resilient elastomeric material.

11. The prophylactic condom recited in claim 10, wherein said resilient elastomeric material is selected from the group including rubber, latex, polyurethane, polyisoprene, polyurethane, or neoprene.

12. A prophylactic condom, comprising:
a. a continuous elastic tubular membrane having a closed distal end, an open proximal end, a head between said closed distal end and said open proximal end, a shaft section between said head and said open proximal end, and a neck section on said shaft section, wherein said tubular membrane creates a sheath extending between said open proximal end to said closed distal end;
b. a bulbous portion integrally formed on said tubular membrane and extending along at least a portion of said tubular membrane along said head from said closed distal end to said neck section, said bulbous portion having a diameter greater than the adjacent said sheath and said neck section whereby said bulbous portion will fold over said neck section to form a sleeve around said sheath; and
c. said tubular membrane and said bulbous portion being integrally formed together from an elastomeric material.

13. The prophylactic condom of claim 12, wherein said continuous elastic tubular membrane is a comprised of a prophylactically suitable elastomeric material.

14. The prophylactic condom of claim 13, wherein said prophylactically suitable elastomeric material includes rubber, latex, polyurethane, polyisoprene, and neoprene.

15. The prophylactic condom of claim 14, further comprising a beaded edge upon said open proximal end whereby said tubular membrane may be rolled and unrolled.

16. The prophylactic condom of claim 15, further comprising a coating of lubricant over said tubular membrane.
